(19) United States
(12) Patent Application Publication
(10) Pub. No.: US 2005/0063990 A1
(43) Pub. Date: Mar. 24, 2005

(54) Compositons and methods for weight loss

(76) Inventor: Robert H. Keller, Hollywood, FL (US)

Correspondence Address: FROMMER LAWRENCE & HAUG, 745 FIFTH AVENUE- 10TH FL., NEW YORK, NY, 10151, US

(21) Appl. No.: 10/664,700

(22) Filed: Sep. 18, 2003

(51) Int. Cl. A61K 31/555; A61K 35/84; A61K 35/78; A61K 35/80

(52) U.S. Cl. 424/195.15; 424/195.17; 514/184; 514/560; 424/725

(57) ABSTRACT

A composition is provided for raising endorphin levels and increasing insulin sensitivity in a mammal.

### FIELD OF THE INVENTION

This invention relates to, inter alia, a composition and a dietary supplement for controlling weight loss in mammals. The invention further relates to methods of controlling weight in mammals.

Documents cited in this text, and all documents cited or referenced in the documents cited in this text, are incorporated herein by reference.

### BACKGROUND OF THE INVENTION

Obesity is a condition that affects millions of Americans. Recent statistics by the Center for Disease Control (“CDC”) show that approximately 61% of all Americans are greater than 10 lbs. overweight. There is also evidence to suggest that the percentage of Americans that are 20-40 lbs. overweight, corresponding to a Body Mass Index (“BMI”) of 25-30 wt. Lbs/ht. m², is increasing. Even mild obesity increases the risk for a number of illnesses such as diabetes, hypertension, and even some forms of cancer. These figures have engendered a 25-30 billion dollar diet industry and yet statistics from the CDC suggest that 50 million U.S. citizens diet per year and less than 5% keep the weight off. Therefore, effective methods for weight reduction are constantly being sought.

This has become a staggering U.S. health problem as 13% of teenagers fall into the obese category (BMI≧30). In addition, the CDC estimates that one third of cancer deaths in 2002 are directly attributable to obesity. Further, over 22% of Americans have metabolic syndromes associated with hypertension, fat abnormalities in the blood such as increased triglycerides, high cholesterol and decreased LDL, and increased heart attacks and cerebral vascular accidents, such as strokes. In fact, the CDC now estimates that 50% of deaths from all causes are associated with obesity with a BMI≧35 and that 52% of deaths from all causes in men and 62% of deaths from all causes in women are associated with a BMI of ≧40. In fact, even a 10-20 lb. increase over ideal body weight increases the risk of death by 20%.

Despite these statistics, estimates suggest that less than three percent of Americans follow the American Dietary Association Recommendations; 40% of Americans do not participate in any physical activity; and the average caloric intake of the U.S. population is 3700 calories, while only 1800-2400 calories are required to maintain a basal metabolic rate.

Although there are many different treatment regimens in use today that can produce short term or temporary weight loss, most are associated with a rapid increase in weight gain once treatment is terminated. Caloric restriction is the main goal of most weight reduction treatment regimens. The basic principle is that if intake of food is less than the energy expenditure, stored calories will be consumed, mainly in the form of fat. However, once the diet regimen is broken, weight is quickly regained.

Other treatment regimens are based on the principle of increasing metabolism or suppressing appetite. By increasing metabolism, calories are burned thereby decreasing body weight. Most weight loss products utilize caffeine or artificial stimulants, as well as sugars or mahuang (ephedra) that act as stimulants to give short-lived appetite suppressant effect. While these products might suppress appetite, these treatments are not effective or have side effects, particularly those involving use of non-prescription and prescription drug products. For example, stimulants promote anxiety and nervousness and raise the heart rate in many mammals. In addition, the FDA has issued data proving the inefficacy of commonly used diet products containing caffeine (guarana), Guar gum, dextrose, diet patches, natural fat blockers such as chitosan, starch blockers, and spirulina. Also, most recently, the FDA has proposed a ban on ephedra because of the deaths associated with its use. These products may be effective for a short time by reducing food intake, but ultimately users of these products return to their former eating habits Thus, there is rapid weight increases once treatment is terminated to a body weight (and frequently more) than what is lost within a year of dieting.

Weight reduction has also been attempted using surgical intervention wherein the size of the stomach is reduced so that a feeling of gastric fullness is produced, resulting in a decrease in appetite and food intake. One such method is placement of a mechanical device such as an inflatable balloon into the stomach. However, such invasive methods are not routinely used.

Hence, there is a need for an effective composition and method for controlling the weight of a mammal without side effects. The present invention provides an effective all-natural weight loss composition and a method for controlling weight without side effects. The present invention further provides a dietary supplement.

### OBJECTS OF THE INVENTION

An object of the present invention is to provide an effective, all-natural composition, which when administered to a mammal produces weight loss through decreasing appetite and improving insulin sensitivity.

Another object of the present invention is to provide a method of reducing weight in mammals that comprises administering to a mammal a composition that decreases appetite and improves insulin sensitivity.

Another object of the present invention is an effective, all-natural dietary supplement composition, which when administered to a mammal decreases appetite and improves insulin sensitivity that produces weight loss.

Additional features and benefits of the invention will become apparent from the detailed description, and claims set forth below.

### SUMMARY OF THE INVENTION

Accordingly a composition is provided that produces weight loss in a mammal comprising an agent capable of raising endorphins levels in a mammal and an agent capable of increasing insulin sensitivity in a mammal and/or an agent which slows digestion.

A method of reducing weight in a patient is also provided comprising administering to a patient a composition that produces weight loss in a mammal comprising an agent capable of raising endorphins levels in a mammal and an agent capable of increasing insulin sensitivity in a mammal and/or an agent which slows digestion.

A dietary supplement composition is also provided comprising an agent to raise endorphin levels in a mammal and an agent to increase insulin sensitivity in a mammal and/or an agent which slows digestion.

### DETAILED DESCRIPTION OF THE PREFERRED EMBODIMENT

The present invention provides for a composition and method for controlling weight loss in mammals. The present invention further provides an all-natural and safe weight loss composition, which when administered to a mammal decreases appetite and increases insulin sensitivity. The composition comprises the combination of an agent that raises endorphin levels and an agent for increasing insulin sensitivity in mammals and/or an agent which slows digestion.

The composition of the present invention utilizes an agent that raises endorphin levels in mammals. Active agents extracted from plants or produced synthetically may be used to raise endorphin levels in mammals. Viscus album is an extract of European mistletoe. European mistletoe is a semiparasite of coniferous and leafy trees. Viscus Album has been used in traditional Chinese medicine for its calming effect, treatment of mental and physical exhaustion, nervousness and anxiety, all of which are ascribed to its ability to increase B endorphins. (Natural Medicines Comprehensive Database, 2003). In addition, its ability to raise B endorphin levels, which interferes with the Central Nervous System (CNS) hunger center(s), derive from studies demonstrating its immunologic properties are similar to B endorphins and especially Metencephalon as it increases interleukin 12, and interleukin 2, stimulates CD₄ positive TH₁ cells and interferon and activate natural killer cells (Stein G. M., Edlund U., Pfuller U., Bussing A., Schietzel M., Influence of polysaccharides form Visum album L. on human lymphocytes, monocytes and granulocytes in vitro., Aniticancer Re 100 September-October; 19(5B):3907-14; Kovaces E., Serum levels of IL-12 and the production of IFN-gamma, IL-2 and IL-4 by peripheral blood mononuclear cells (PBMC) in cancer patients treated with Viscum album extract., Biomed Pharmacother, 54(6):305-10 (2000); Stein G M., Pfuller U., Schietzel M., Bussing A., Intercellular expression IL-4 and inhibition of IFN gamma by extracts from European mistletoe is related to induction of apoptosis, Anticancer Res September-October; 20(5A):2987-94 (2000)) all of which are similar to the reported results of increasing B endorphins. Viscus album, when administered to a mammal, raises B endorphin levels and results in decreased appetite, similar to loss of hunger associated with prolonged exercise. The raised endorphin levels decrease appetite thereby decreasing food intake.

The composition of the present invention further comprises an agent to increase insulin sensitivity and/or an agent which slows digestion. One example is Tremella mushrooms. Tremella mushrooms provide 60% protein and 40% complex carbohydrates which slows digestion as well as a number of trace minerals to increase insulin sensitivity in mammals. In addition, Tremella mushrooms have 6.2% soluble dietary fiber and 52.3% insoluble dietary fiber including TFB. Tremella mushrooms slow digestion, increases insulin sensitivity, and is reported to significantly decrease LDL cholesterol, hepatic total cholesterol and triglycerides. (Cheng H H., Hou W C., Lu M L., Interactions of lipid metabolism and intestinal physiology with Tremella fuciformis Berk edible mushroom in rats fed in high-cholesterol diet with or without Nebacitin, Agric Food Chem,;50(25):7438-43 (2002); Kiho T., Kochi M., Usui S., Hirano K., Airawa K., Inakuma T., Antidiabetic effect of an acidic polysaccharide (TAP) from Tremella aurantia and its degradation product (TAP-H), Biol Pharm Bull, 24(12)1400-3 (2001)). In addition, at least one acidic polysaccharide (TAP) from Tremella significantly lowers insulin levels and decreases plasma levels of lipoperoxide, which in effect lowers insulin resistance, a universal mechanism in abdominal obesity. (Kiho T., Kobayashi T., Morimoto H., Usui S., Ukai S., Hirano K., Aizawa K., Inakuma T., Structural features of an anti-diabetic polysaccharide (TAP) from Tremella aurantia, Chem Pharm Bull (Tokyo), 48(11):1793-5 (2000); Kiho T., Morimoto H., Kobayashi T., Usui S., Ukai S., Aizawa K., Inakuma T., Effect of a polysaccharide (TAP) from the fruiting bodies of Tremella aurantia on glucose metabolism in mouse liver, Biosci Biotechnol Biochem, 64(2):417-9 (2000)). Furthermore, lectin from Tremella POL is an appetite suppressant. In fact, a diet containing 0.1% of POL decreased food intake by 50% in animals. (Kawagishi H., Suzuki H., Watanabe H., Nakamura H, Sekiguchi T., Urata T., Usui T., Sugiyama K., Suganuma H., Nakuma T., Ito K., Hashimoto Y., Ohnishi-Kameyama M., Nagata T., A lectin from an edible mushroom Pleurotus streatus as a food intake-suppressing substance, Biochem Biophys Acta, 147(3):299-308 (2000)).

In addition to Viscus album and Tremella, the composition of the present invention may include radioiodine-free kelp, which provides 60% protein and 40% complex carbohydrates. The amount of kelp prevents starvation and prevents utilization of muscle tissue for energy. Studies have shown that kelp increases glutathione perodidase, which decreases lipid peroxidation, thus improving liver function (i.e., reduced glutathione) and improving the ability of fat to be utilized as fuel rather than being stored. (Maruyama H., Watanabe K., Yamamoto I., Effect of dietary kelp on lipid peroxidation and glutathione peroxidase activity in livers of rats given breast carcinogen DMBA, Nutr Cancer, 15(3-4):221-8 (1991)). In another study, kelp was shown to contain 20.5% protein, 1.7% fat and 45% complex carbohydrates with high available to ingested energy (net energy available 62%) (Tajiri J., Higashi K,, Morita M,, Umeda T,, Sato T., Studies of hypothyroidism in patients with high iodine intake, J. Clin Endocrinol Metab, 63(2):412-7/(1986)) and has been shown to be a good source of iodine, which maximizes thyroid function. (Teas J., The consumption of seaweed as a protective factor in the etiology of breast cancer, Med Hypotheses, 7(5):601-13 (1981)). Furthermore, kelp reduces plasma cholesterol and is hydrophilic which together with Tremella creates a peripheral sense of satiety while Viscus album creates a general sense of satiety through its production of B endorphins.

Two additional supplements may be also included in the present composition, both for their synergistic effects with Tremella and kelp, as well as independent additive effects. The first is conjugated linoleic acid (CLA). CLA decreases fat and increases lean body mass by improving the efficient use of free fatty acids as fuel. CLA enhances muscle growth and is effective in eliminating lipogenesis (creation of fat cells) and inhibits existing fat cell size from increasing. CLA has been shown to decrease adipose number (Experimental Biology Meeting, New Orleans, April 20-24, (2002), FASEB of Scientific Meeting and Conferences) and decrease abdominal fat among men classified as abdominally obese (Riserus U., Berglund I., Vessby B., Conjugated linoleic acid (CL) reduced abdominal adipose tissue in obese middle-aged men with signs of the metabolic syndrome: a randomized controlled trial, Int J. Obes Relat Metab Disord, 25(8)1129-35 (2001)) and reduces total fat mass in overweight as well as obese humans. (Blankson H., Stakkestad J A., Fagertun H., et al. Conjugated linoleic acid reduces body fat mass in overweight and obese humans, J. Nutr; 120(12):2943-8 (2000)). In addition, in animal studies, CLA has been shown to decrease the amount of ingested food stored as body fat, and increase the use of fat as fuel (energy expenditure) by 74% (Terpstra A H., Beynen A C., Everts H., et al. The decrease in body fat in mice fed conjugated linoleic acid is due to increases in energy expenditure and energy loss in the excreta, J Nutr, 1322(5):940-5 (2002)) and increase lean body mass by promoting free fatty acids as an energy source for muscles. (Ostrowska E., Muralitharan M., Cross R F., et al. Dietary conjugated linoleic acids increase lean tissue and decrease fat deposition in growing pigs, J Nutr, 129(11):2037-42 (1999)).

In addition, chromium niconalate may be added to the composition, which works with insulin in the metabolism of sugar. Chromium niconalate promotes glucose utilization and has been shown to be a necessary factor for maximum insulin function. Insulin requires chromium to function properly. Chromium strengthens certain effects of insulin on the body's cell by making insulin that is present function more effectively in the cells of the body. Although chromium niconalate is specifically mentioned, it is contemplated that other agents that have chromium ions are also useful.

The composition of the present invention may be orally administered with eight ounces of iced green tea or iced water to a mammal. The results of such an administration include, for example:
1. peripheral and central mechanisms for satiety;
2. slows digestion;
3. reduces adipocyte size and numbers;
4. provides significant increased energy utilization;
5. maximizes thyroid function;
6. promotes insulin sensitivity via a number of mechanisms;
7. normalizes leptin abnormalities associated with insulin resistance and being overweight (BMI>25) or obese (BMI>30); and
8. promotes the utilization of free fatty acids as energy.

The composition of the present invention provides a combination of selected agents that has been shown to be effective in producing weight loss in pilot studies.

Embodiments in accordance with the invention may also include dietary supplements that are particularly useful in nutrition and weight loss. The dietary supplement, in addition to the claimed elements, may also include amino acids, minerals, vitamins, herbs, and any other nutrients.

Table I presents one embodiment of the composition of the invention.

TABLE I
The components per 6 capsules include:

| | | | |
|---|---|---|---|
| 1. | Viscus album (Stem) 4:1 extract | (Minstar) | 1000 mg. |
| 2. | Kelp | (Minstar) | 1000 mg. |
| 3. | Tremella mushroom | (Minstar) | 500 mg. |
| 4. | Conjugated linoleic acid | (Pharm Grade) | 1000 mg. |
| 5. | Chromium Niconalate | | 2 mg. |

The amounts of the components can increase two to three fold due to lack of known side effects except for Chromium and Viscus Album. In addition, the composition may not be suitable for pregnant or breastfeeding mothers, immunocompromized patients, patients with active cancer, patients with a known allergy to mushrooms or iodine and/or patients with autoimmune thyroiditis.

The composition may be in any convenient dietary supplement form. Examples include capsules, wherein, for example, a serving size is two capsules. To make capsules, the above components are mixed with commonly used food stuffs or an inert carrier (e.g., starch or calcium carbonate or lactose) in a conventional manner as known in the art. It is to be appreciated that other suitable forms, such as powder or liquid, prepared in conventional manners, with or without food stuff or inert carriers, are also contemplated.

The combination of the aforesaid ingredients provides maximum results in weight loss. The combination produces results that were heretofore unachievable with the weight loss supplements in the prior art.

Thus, the above composition describes an embodiment of the composition of the invention that decreases appetite, increases insulin sensitivity, metabolizes fat, and converts stored body fat into energy.

### EXAMPLES

A 90-day study was conducted to investigate the effect of the composition of the invention on weight loss.

A test panel was selected of 5 individuals who were otherwise healthy with a BMI between 25 and 30 kg/M². None of the subjects were asked to change their diet or exercise routine (if any).

The components in Table I were mixed and prepared as capsules. The dose of 2 capsules was taken with 8 oz of iced decaffeinated green tea or iced water a half hour before each of three meals.

The total amount per day of components in the capsules of the tested embodiment of the invention contained: about 1000 mg Viscus Album (Stem) 4:1; about 1000 mg Kelp; about 500 mg Tremella mushroom; about 1000 mg Conjugated linoleic acid and about 2 mg Chromium Niconalate. The subjects taking the composition reported no side effects.

Members of the study taking the composition lost an average of 10 pounds per month over the course of the 90 day trial. The data obtained suggests that the composition of the invention is effective in reducing feelings of hunger and therefore reduced overeating in overweight individuals. The study also indicates that the composition of the invention increases insulin sensitivity. The results of these experiments demonstrate the effectiveness of the combination in weight reduction in mammals.

One theory proposed for the effects of one embodiment of the composition of the invention as part of a weight loss regimen is that the effects noted above are due to the ability of the components of the composition of the invention acting in concert or synergistically.

As will be obvious to those of skill in the art, other herbal extracts or agents having these active agents can also be selected for use in a composition of the present invention. Herbal extracts or agents for combination into a composition of the present invention are obtained in accordance with methods well known and routine to those of skill in the art.

Although preferred embodiments of the present invention and modifications thereof have been described in detail herein, it is to be understood that this invention is not limited to those precise embodiments and modifications, and that other modifications and variations may be affected by one skilled in the art without departing from the spirit and scope of the invention as defined by the appended claims.

### References

Preuss H G., Bagchi D., Bagchi M., Protective effects of a novel niacin-bound chromium complex and a grape seed proanthocyanidin extract on advancing age on various aspects of syndrome X, Ann NY Acad Sci., 957:250-9 (2002).
Thomas V L., Gropper S S., Effect of chromium nicotinic acid supplementation on selected cardiovascular disease risk factors, Biol Trace Elem Res., 55(3):297-305 (1996).
Urberg M, Zemel M B., Evidence for synergism between chromium and nicotinic acid in control of glucose tolerance in elderly humans, Metabolism;36(9):869-9 (1987).

1. A composition that produces weight loss in a mammal, comprising an agent capable of raising endorphin levels in a mammal and an agent capable of increasing insulin sensitivity in a mammal.

2. The composition of claim 1, wherein the agent capable of raising endorphin levels in a mammal comprises an herbal extract.

3. The composition of claim 2, wherein the herbal extract is viscus album.

4. The composition of claim 1, wherein the agent capable of increasing insulin sensitivity in a mammal comprises chromium ions.

5. The composition of claim 4, wherein the agent capable of increasing insulin sensitivity in a mammal is chromium niconalate.

6. The composition of claim 4, wherein the agent capable of increasing insulin sensitivity in a mammal comprises Tremella mushrooms.

7. The composition of claim 1, further comprising an agent capable of slowing digestion.

8. The composition of claim 7, wherein said agent capable of slowing digestion comprises Tremella mushrooms.

9. The composition of claim 1, further comprising conjugated linoleic acid.

10. The composition of claim 1, further comprising kelp.

11. A composition that produces weight loss in a mammal, comprising an agent capable of raising endorphin levels in a mammal and an agent capable of slowing digestion in a mammal.

12. The composition of claim 1 1, wherein the agent capable of raising endorphin levels in a mammal comprises an herbal extract.

13. The composition of claim 12, wherein the herbal extract is viscus album.

14. The composition of claim 1 1, wherein the agent capable of slowing digestion in a mammal comprises Tremella mushrooms.

15. The composition of claim 11, further comprising an agent capable of increasing insulin sensitivity in a mammal.

16. The composition of claim 15, wherein the agent capable of increasing insulin sensitivity comprises Tremella mushrooms.

17. The composition of claim 15, wherein the agent capable of increasing insulin sensitivity comprises chromium ions.

18. The composition of claim 16, wherein the agent capable of increasing insulin sensitivity is chromium niconalate.

19. The composition of claim 11, further comprising conjugated linoleic acid.

20. The composition of claim 11, further comprising kelp.

21. A composition that produces weight loss in a mammal comprising:
an effective amount of a viscus album;
an effective amount of chromium niconalate;
an effective amount of Tremella mushrooms;
an effective amount of conjugated linoleic acid; and
an effective amount of kelp.

22. A method of reducing weight in a patient, comprising administering to said patient a composition of claim 1.

23. The method of claim 22, wherein the agent of claim 1 capable of raising endorphin levels in a mammal is an herbal extract.

24. The method of claim 23, wherein the herbal extract is viscus album.

25. The method of claim 22, wherein the agent of claim 1 capable of increasing insulin sensitivity in a mammal comprises chromium ions.

26. The method of claim 25, wherein the agent capable of increasing insulin sensitivity in a mammal is chromium niconalate.

27. The method of claim 22, wherein the agent of claim 1 capable increasing insulin sensitivity in a mammal comprises Tremella mushrooms.

28. The method of claim 22, wherein the composition further comprises an agent capable of slowing digestion.

29. The method of claim 28, wherein the agent capable of slowing digestion comprises Tremella mushrooms.

30. The method of claim 22, wherein the composition further comprises conjugated linoleic acid.

31. The method of claim 22, wherein the composition further comprises kelp.

32. The method of claim 22, wherein the composition promotes prevents of muscle tissue for energy.

33. A method of reducing weight in a patient, comprising administering to a patient a composition of claim 11.

34. The method of claim 33, wherein the agent of claim 11 capable of raising endorphin levels in a mammal comprises an herbal extract.

35. The method of claim 34, wherein the herbal extract is viscus album.

36. The method of claim 33, wherein the agent of claim 11 capable of slowing digestion comprises Tremella mushrooms.

37. The method of claim 33, wherein the composition further comprises an agent capable of increasing insulin sensitivity in a mammal.

38. The method of claim 37, wherein the agent capable of increasing insulin sensitivity in a mammal comprises Tremella mushrooms.

39. The method of claim 37, wherein the agent capable of increasing insulin sensitivity comprises chromium ions.

40. The method of claim 39, wherein the agent capable of increasing insulin sensitivity is chromium niconalate.

41. The method of claim 33, wherein the composition further comprises conjugated linoleic acid.

42. The method of claim 33, wherein the composition further comprises kelp.

43. The method of claim 33, wherein the composition prevents utilization of muscle tissue for energy.

44. A dietary supplement composition comprising an agent capable of raising endorphin levels in a mammal and an agent capable of increasing insulin sensitivity in a mammal.

45. A dietary supplement composition comprising an agent capable of raising endorphin levels in a mammal and an agent capable of slowing digestion in a mammal.

46. A dietary supplement composition for controlling weight loss comprising:
an effective amount of a viscus album;
an effective amount of chromium niconalate;
an effective amount of Tremella mushrooms;
an effective amount of conjugated linoleic acid; and
an effective amount of kelp.
